(19) United States
(12) Patent Application Publication
(10) Pub. No.: US 2003/0204239 A1
(43) Pub. Date: Oct. 30, 2003

(54) Endovascular stent with a preservative coating

(76) Inventors: Wenda Carlyle, Irvine, CA (US); Eugene Tedeschi, Santa Rosa, CA (US)

Correspondence Address: MEDTRONIC AVE, INC., 3576 UNOCAL PLACE, SANTA ROSA, CA, 95403, US

(21) Appl. No.: 10/133,181

(22) Filed: Apr. 26, 2002

(51) Int. Cl. A61F 2/06

(52) U.S. Cl. 623/1.11; 623/1.42

(57) ABSTRACT

The present invention provides a system for treating a vascular condition, including a catheter, a stent including a stent framework coupled to the catheter, a preservative coating operably disposed on the stent framework, wherein the preservative coating includes at least one antioxidant.

### FIELD OF THE INVENTION

This invention relates generally to biomedical stents. More specifically, the invention relates to a preservative coating containing at least one antioxidant on a stent framework.

### BACKGROUND OF THE INVENTION

The efficacy of endovascular stents may be increased by the addition of polymeric stent coatings that contain pharmaceutical drugs. These drugs may be eluted from the stent coating when in the body, delivering their patent effects in the tissue bed surrounding the implanted stent. The effectiveness of these drugs may be improved because the localized levels of the medications may be higher and potentially more effective than orally or intravaneously delivered drugs that distribute throughout the body, and which may have little effect on the impacted area or may be expelled rapidly from the body without reaching their pharmaceutical intent. Drug release from tailored stent coatings may have controlled, timed-release qualities, eluting their bioactive agents over hours, weeks or even months.

Unfortunately, drug polymers may not provide maximal pharmaceutical benefit due to degradation of the drug within the polymer or from degradations of the polymer coating prior to insertion into the body. Degradations of the drug or polymer may impact the delivery rate of the drug. The drug may elute its pharmacologically active constituents too quickly or too slowly. If a drug is eluted too quickly, it may be ineffective and possibly toxic. If a drug is eluted too slowly, then its intended effect on the body may be compromised.

Degradation of the polymer coating or the drugs interdispersed within the polymer-drug coating may occur with prolonged exposure to light and air, as the constituents of the drug polymer may oxidize or the molecular chains may scission. Furthermore, the coating may crystallize, crack, or fall off during assembly, packaging, storage, shipping, preparation and sterilization prior to deployment unless effectively stabilized. Stabilization of the drug-polymer coating may aid in the control of the bioavailability of the therapeutic components to maximize effectiveness.

Stabilized drug polymer coatings may have a tendency to corrode an underlying metallic stent, or to degrade a non-metallic stent. A method to inhibit or prevent the stabilized drug polymer coating from degrading the stent framework, while improving the metal-adhering characteristics would be beneficial.

It is an object of this invention, therefore, to provide a system for treating heart disease and other vascular conditions using stabilized drug-eluting stents, to provide a method for inhibiting the corrosion of metallic stents or degradation of polymeric stents when using drug polymers, to provide methods of manufacturing stabilized drug-polymer coated stents, to ensure the quality and performance of polymer-drug coatings on cardiovascular stents and other implanted devices, and to overcome the deficiencies and limitations described above.

### SUMMARY OF THE INVENTION

One aspect of the invention provides a system for treating a vascular condition, including a catheter, a stent including a stent framework coupled to the catheter, and a preservative coating disposed on the stent framework. The preservative coating includes at least one antioxidant.

The preservative coating may include a drug polymer with a bioactive agent to provide a therapeutic characteristic. The bioactive agent may include an antineoplastic agent, an antiproliferative agent, an antisense agent, an antiplatelet agent, an antithrombogenic agent, an anticoagulant, an antibiotic, an anti-inflammatory agent, a gene therapy agent, an organic drug, a pharmaceutical compound, a recombinant DNA product, a recombinant RNA product, a collagen, a collagenic derivative, a protein, a protein analog, a saccharide, a saccharide derivative, or a combination thereof. The preservative may include butylated hydroxytoluene, vitamins A, B, C, D, or E, or other suitable antioxidant.

The catheter may include a balloon used to expand the stent, or include a sheath that retracts to allow expansion of the stent. The stent framework may include a metallic base including stainless steel, nitinol, tantalum, MP35N alloy, a suitable biocompatible alloy, a suitable biocompatible material, or a combination thereof. The stent framework may include a polymeric base.

A barrier coating may be interdispersed between the stent framework and the preservative coating. The barrier coating may include parylene or a silane coupling agent. The barrier coating may have a thickness between 0.1 microns and 10 microns.

Another aspect of the invention is a preservative-coated stent, including a stent framework, a polymeric coating on the stent framework and a preservative interdispersed within the polymeric coating. The preservative may include vitamin A, vitamin B, vitamin C, vitamin D, vitamin E, butylated hydroxytoluene, a suitable antioxidant, or combinations thereof. The preservative-coated stent may include a barrier coating interdisposed between the stent framework and the preservative coating. The barrier coating may include parylene, a silane coupling agent, a suitable corrosion-resistant material, or combinations thereof.

Another aspect of the invention is a method of manurfacturing a preservative-coated stent, including the steps of mixing a polymeric material with a solvent to form a polymeric mixture, interdispersing a preservative in the polymeric mixture to form a preservative coating, applying the preservative coating onto the stent framework, and drying the preservative coating. The preservative may include at least one antioxidant, such as vitamin A, vitamin B, vitamin C, vitamin D, vitamin E, or butylated hydroxytoluene.

A barrier coating may be applied onto the stent framework prior to the application of the preservative coating. The barrier coating may include parylene, a silane coupling agent, or other suitable barrier coating material.

The aforementioned, and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

Drug polymer coatings on endovascular stents may be stabilized using preservatives. The present invention improves the quality and efficacy of drug-polymer coated stents through the use of preservatives in either the drug or the polymer. A system for treating various vascular conditions using drug-polymer coated stents with a preservative coating is described, along with a barrier coating to provide corrosion protection for the stent framework and a method of manufacturing a preservative-coated stent.

The present invention is illustrated by the accompanying drawings of various embodiments and the detailed description given below. The drawings should not be taken to limit the invention to the specific embodiments, but are for explanation and understanding. The foregoing aspects and other attendant advantages of the present invention will become more readily appreciated by the detailed description taken in conjunction with the accompanying drawings, wherein:

FIG. 1 is an illustration of one embodiment of a system for treating a vascular condition containing a catheter, a stent, and a preservative coating on the stent, in accordance with the current invention;

FIG. 2 is an illustration of a stent cross-section containing a preservative coating on the stent surface, in accordance with the current invention;

FIG. 3 is an illustration of a stent cross-section with a preservative coating on the stent surface with an interdisposed barrier coating between the preservative coating and the stent framework, in accordance with the current invention;

FIG. 4 is a flow diagram of one embodiment of a method for manufacturing a preservative coated stent with a corrosion-resistant coating, in accordance with the current invention.

### DETAILED DESCRIPTION OF THE PRESENTLY PREFERRED EMBODIMENTS

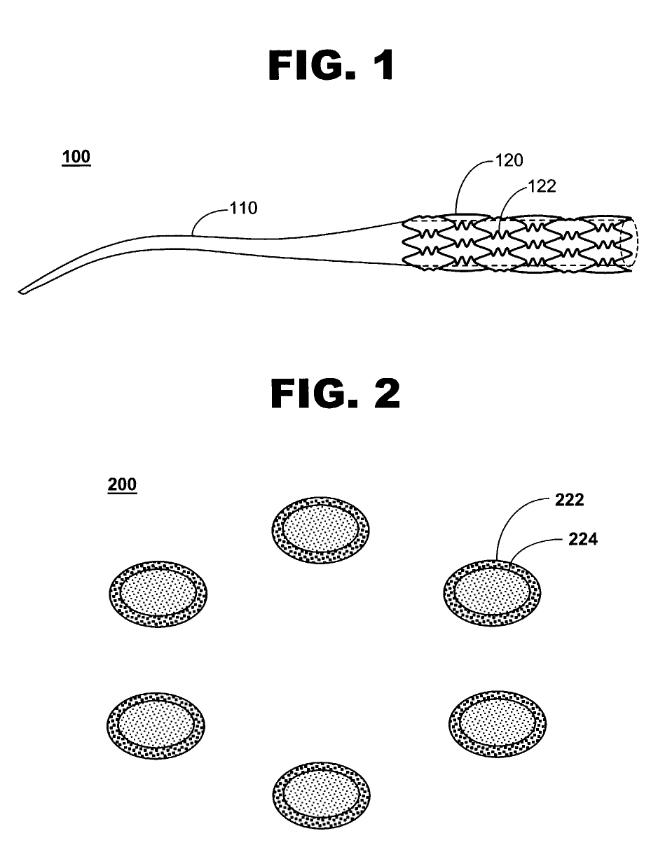

One aspect of the present invention is a system for treating coronary heart disease and other vascular conditions, using catheter-deployed endovascular stents with polymeric coatings including one or more drugs with desired timed-release properties and a preservative containing at least one antioxidant. Treatment of vascular conditions may include the prevention or correction of various ailments and deficiencies associated with the cardiovascular system, urinogenital systems, biliary conduits, abdominal passageways and other biological vessels within the body. One embodiment of the system for treating vascular conditions, in accordance with the present invention, is illustrated in FIG. 1 at 100. Vascular condition treatment system 100 may include a catheter 110, a stent 120 coupled to the catheter, and a preservative coating 122 with an interdispersed preservative on the stent or stent framework. Preservative coating 122 may include one or more drugs and at least one antioxidant. Each drug may include a bioactive agent. The bioactive agent may be a pharmacologically active drug or bioactive compound. The bioactive agent may be eluted from the preservative coating when the stent has been deployed in the body. Elution refers to the transfer of the bioactive agent out from preservative coating 122. The elution rate is determined by the rate at which the bioactive agent is excreted from preservative coating 122 into the body, typically measured in weight per unit time, or in weight per unit time per peripheral area of the stent. The composition of the preservative coating and the interdispersed drugs may control the elution rate of the bioactive agent. The preservative coating may include between less than one to greater than seventy-five percent of the bioactive drug by weight.

Control of the elution rate of the bioactive agent may be achieved by increasing the effective molecular weight of the bioactive agent and thereby slowing the diffusion of the pharmaceutical drug from the preservative coating, by modifying the drug to decrease the effective solubility of the bioactive agent in the body with the addition of less soluble attachments, by adding attachments that slow the metabolization of the bioactive agent; by careful selection or appropriate modifications of the polymer coating, or by any combination of the above.

Many drugs and polymers are unstable and subject to degradation during processing, packaging, sterilization, or storage of a drug-polymer coated stent. During sterilization, for example, oxidation of the drug or polymer may occur resulting in hydrolytic damage, cleavage of the polymeric bonds, and breakdown of the polymer and/or drug. The lack of drug stability may cause decreased efficacy, and in some cases increased toxicity of the stent. The present invention solves this problem through the use of an effective amount of preservatives in either the drug or polymer of a drug coated stent so as to reduce or prevent drug and polymer degradation. For example, degradation due to oxidation may be reduced with addition of antioxidants. Examples of preservatives that may be used include antioxidants such as butylated hydroxytoluene (BHT) or vitamins A through E.

Upon insertion of catheter 110 and stent 120 with preservative coating 122 into a directed vascular region of a human body, stent 120 may be expanded by applying pressure to a suitable balloon inside the stent, or by retracting a sheath to allow expansion of a self-expanding stent. Balloon deployment of stents and self-expanding stents are well known in the art. Catheter 110 may include the balloon used to expand stent 120. Catheter 110 may include a sheath that retracts to allow expansion of the stent.

The preservative may be interdispersed within preservative coating 122, and may be eluted then metabolized or discarded by the body.

FIG. 2 shows an illustration of a stent cross-section containing a drug-polymer with at least one preservative on the stent surface, in accordance with the present invention at 200. The drug-polymer or polymeric coating may also be referred to herein as a preservative coating. Drug-polymer coated stent 200 with an interdispersed preservative may include a preservative coating 222 on a stent framework 224. Preservative coating 222 may contain one or more pharmaceutical drugs. Preservative coating 222 may contain a polymeric matrix in which one or more pharmaceutical drugs are interdispersed. One or more preservatives may be interdispersed within preservative coating 222.

The preservatives may include one or more antioxidants. Examples of preservatives that may be used include antioxidants such as butylated hydroxytoluene (BHT), vitamin A, vitamin B, vitamin C, vitamin D, vitamin E, or other anti-oxidant nutrient or agent. Oxygen may react preferentially with BHT or other antioxidants rather than degrade the polymer or drug, thereby protecting the polymer drug.

The drugs and one or more antioxidants may be encapsulated in a polymer coating as a microbead, microparticle or nanoencapsulation technology with albumin, liposome, ferritin or other biodegradable proteins and phospholipids, prior to application on the stent.

Stent framework 224 may include a metallic or polymeric base. Stent framework 324 may include a base material of stainless steel, nitinol, tantalum or an MP35N alloy. The stent or stent framework may include a base material of a suitable biocompatible alloy, a suitable biocompatible material including a biodegradable polymeric material, or a combination thereof.

The bioactive agent may include an antineoplastic agent such as triethylene thiophosphoramide, an antiproliferative agent, an antisense agent, an antiplatelet agent, an antithrombogenic agent, an anticoagulant, an antibiotic, an anti-inflammatory agent, a gene therapy agent, an organic drug, a pharmaceutical compound, a recombinant DNA product, a recombinant RNA product, a collagen, a collagenic derivative, a protein, a protein analog, a saccharide, a saccharide derivative, or combinations thereof.

The bioactive agent may be any therapeutic substance that provides a therapeutic characteristic for the prevention and treatment of disease or disorders. An antineoplastic agent may prevent, kill, or block the growth and spread of cancer cells in the vicinity of the stent. An antiproliferative agent may prevent or stop cells from growing. An antisense agent may work at the genetic level to interrupt the process by which disease-causing proteins are produced. An antiplatelet agent may act on blood platelets, inhibiting their function in blood coagulation. An antithrombogenic agent may actively retard blood clot formation. An anticoagulant may delay or prevent blood coagulation with anticoagulant therapy, using compounds such as heparin and coumarins. An antibiotic may kill or inhibit the growth of microorganisms and may be used to combat disease and infection. An anti-inflammatory agent may be used to counteract or reduce inflammation in the vicinity of the stent. A gene therapy agent may be capable of changing the expression of a person's genes to treat, cure or ultimately prevent disease. An organic drug may be any small-molecule therapeutic material. A pharmaceutical compound may be any compound that provides a therapeutic effect. A recombinant DNA product or a recombinant RNA product may include altered DNA or RNA genetic material. Bioactive agents of pharmaceutical value may also include collagen and other proteins, saccharides, and their derivatives.

For example, the bioactive agent may be selected to inhibit vascular restenosis, a condition corresponding to a narrowing or constriction of the diameter of the bodily lumen where the stent is placed. The bioactive agent may generally control cellular proliferation. The control of cell proliferation may include enhancing or inhibiting the growth of targeted cells or cell types.

The bioactive agent may be an agent against one or more conditions including coronary restenosis, cardiovascular restenosis, angiographic restenosis, arteriosclerosis, hyperplasia, and other diseases and conditions. For example, the bioactive agent may be selected to inhibit or prevent vascular restenosis, a condition corresponding to a narrowing or constriction of the diameter of the bodily lumen where the stent is placed. The bioactive agent may generally control cellular proliferation. The control of cell proliferation may include enhancing or inhibiting the growth of targeted cells or cell types.

The bioactive agent may include podophyllotoxin, etoposide, camptothecin, a camptothecin analog, mitoxantrone, rapamycin, and their derivatives or analogs. Podophyllotoxin is an organic, highly toxic drug that has antitumor properties and may inhibit DNA synthesis. Etoposide is an antineoplastic that may be derived from a semi-synthetic form of podophyllotoxin to treat monocystic leukemia, lymphoma, small-cell lung cancer, and testicular cancer. Camptothecin is an anticancer drug that may function as a topoisomerase inhibitor. Related in structure to camptothecin, a camptothecin analog such as aminocamptothecin may be used as an anticancer drug. Mitoxantrone is also an important anticancer drug, used to treat leukemia, lymphoma, and breast cancer. Rapamycin or sirolimus is a medication that may interfere with the normal cell growth cycle and may be used to reduce restenosis. The bioactive agent may also include analogs and derivatives of these agents. Antioxidants may be beneficial on their own rights for their antirestonetic properties and therapeutic effects.

Preservative coating 222 may soften, dissolve or erode from the stent to elute at least one bioactive agent. This elution mechanism may be referred to as surface erosion where the outside surface of the preservative coating dissolves, degrades, or is absorbed by the body; or bulk erosion where the bulk of the preservative coating biodegrades to release the bioactive agent. Eroded portions of the preservative coating may be absorbed by the body, metabolized, or otherwise expelled.

The pharmaceutical drug may separate within preservative coating 222 and elute the bioactive agent. Alternatively, the pharmaceutical drug may erode from stent 120 and then separate into the bioactive agent. The preservative may be eluted and absorbed or expelled by the body. Preservative coating 222 may include multiple pharmaceutical drugs, and more than one preservative. Preservative coating 222 may include a single bioactive agent with various preservatives stabilize the bioactive agent.

Preservative coating 222 may also include a polymeric matrix. For example, the polymeric matrix may include a caprolactone-based polymer or copolymer, or various cyclic polymers. The polymeric matrix may include various synthetic and non-synthetic or naturally occurring macromolecules and their derivatives. The polymeric matrix may include biodegradable polymers such as polylactide (PLA), polyglycolic acd (PGA) polymer, poly (e-caprolactone) (PCL), polyacrylates, polymethacryates, or other copolymers. The pharmaceutical drug may be dispersed throughout the polymeric matrix. The pharmaceutical drug or the bioactive agent may diffuse out from the polymeric matrix to elute the bioactive agent. The pharmaceutical drug may diffuse out from the polymeric matrix and into the biomaterial surrounding the stent. The bioactive agent may separate from within preservative coating 222 and diffuse out from the polymeric matrix into the surrounding biomaterial.

The polymeric matrix may be selected to provide a desired elution rate of the bioactive agent. The pharmaceutical drugs may be synthesized such that a particular bioactive agent may have two different elution rates. A bioactive agent with two different elution rates, for example, would allow rapid delivery of the pharmacologically active drug within twenty-four hours of surgery, with a slower, steady delivery of the drug, for example, over the next two to six months. The preservatives may be selected to stabilize the rapidly deployed bioactive agents and to stabilize the slowly-eluting pharmaceutical drugs.

BHT and other antioxidants and preservatives have known corrosion activity on stainless steel and other metals. Another aspect of the present invention provides a barrier coating prior to deposition of the preservative coating that contains these preservatives to prevent erosion of the metallic base. The barrier coating may have a reactive moiety, or merely an encapsulant laid down around the metal and underneath the drug preservative layer so as to prevent corrosion of the underlying stent.

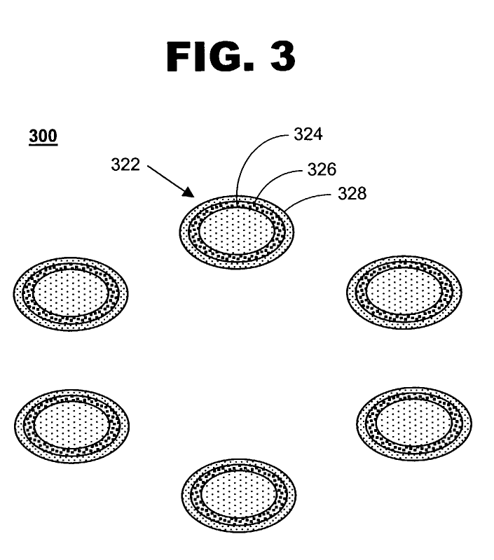

FIG. 3 shows an illustration of a stent cross-section comprising a polymeric coating containing a preservative coating on the corrosion-resistant barrier coating between the preservative coating and the stent framework, in accordance with another embodiment of the present invention at 300. Drug-polymer coated stent 300 with a polymeric coating 322 includes a barrier coating 326 on a stent framework 324 and a preservative coating 328 on barrier coating 326. Preservative coating 328 includes at least one preservative. Preservative coating 328 may optionally include one or more interdispersed bioactive agents. One or more bioactive agents may be interdispersed within preservative coating 328 along with the preservatives. Barrier coating 326 may be void or nearly void of pharmaceutical drugs and preservatives.

Barrier coating 326 may be selected to improve the adhesion and minimizing the likelihood of delamination of the preservative coating from stent framework 324, and to inhibit any corrosive characteristics of preservative coating 328 from degrading stent framework 324. Metal-adhering attributes may aid in the cohesiveness of the preservative coating to metallic stents.

Barrier coating 326 may be comprised of any suitable barrier material that enhances adhesion between preservative coating 328 and stent framework 324 while preventing corrosion of the stent framework. The corrosion-resistant barrier coating may have a predominantly hydrophilic characteristic to improve metal adhesion.

One suitable barrier material is parylene, a conformal protective coating material generally utilized to provide protection and corrosion resistance for coated components. Parylene may be applied at room temperature with deposition equipment that allows a suitable dimer to be vaporized under vacuum and heated to generate a dimeric gas, which is then pyrolized to cleave the dimer into its monomeric form and conformally deposit on the stent framework as a generally transparent polymeric film with thickness less than one micron to greater than several thousandths of an inch.

Another suitable barrier material is a silane-based coating. A silane coupling agent may be used to enhance adhesion of the preservative coating, while protecting the underlying metallic base of the stent framework from any corrosive properties of the preservative coating. Silane coupling agents may be used as corrosion-inhibiting pretreatments. A non-functional silane such as bis-1,2-(triethoxysilyl)ethane (BTSE) or similar molecules with a bis or tris silyl functional silane without an organic function group such as gamma-aminopropyl silane (gamma-APS), or a functional silane that is a trialkoxyesters may be used on a metallic base.

A good silane film for corrosion protection should be covalently bonded to the stent framework through hydrolytically stable metallosiloxane bonds, solidly anchored to the metal by Si—O-metal bonds formed from metal-OH and Si—OH groups. The barrier coating may be less than 0.05 microns up to and exceeding 10 microns thick.

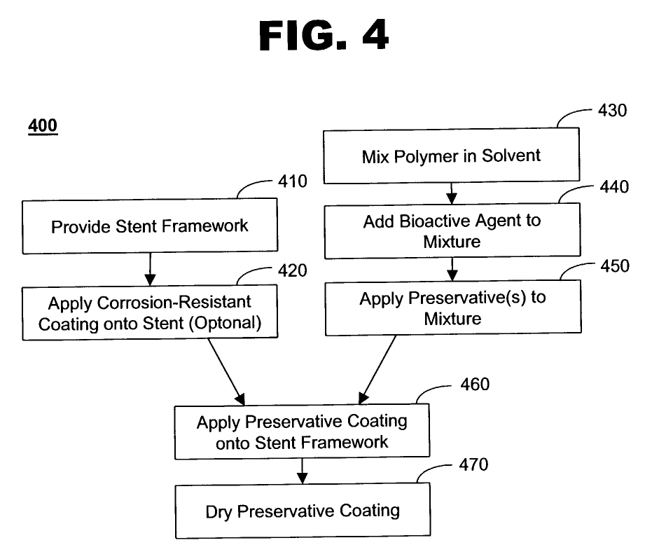

Another aspect of the current invention is a method of manufacturing a drug-polymer stent with a preservative coating. FIG. 4 shows a flow diagram of one embodiment of a method for manufacturing a drug-polymer stent including a corrosion-resistant coating, in accordance with the present invention at 400.

The drug-polymer coated stent with preservatives and a barrier coating may be manufactured by providing a suitable metallic or non-metallic stent framework, as seen at block 410. A corrosion-resistant barrier coating optionally may be applied to the stent framework, as seen at block 420. The barrier coating may be a parylene film, deposited using vacuum deposition methods common in the art. The barrier coating may be a silane coupling agent. The silane coupling agent may be applied on top of a clean metal surface, prior to application of the preservative coating. One preferred method of applying the silane coupling agent is to hydrolyze a dilute solution of the silane in water. The ethoxy or methoxy esters may be hydrolyzed, for example, by mixing water in a 90/10 water/silane ratio by volume. An alcohol such as methanol or ethanol may be used in the solution to increase the solubility of the silane coupling agent in water. The solution may be applied to the stent framework by dipping, spraying or brushing. Excess liquid may be blown off and the film dried in air or at slightly elevated temperatures. Dipping times may be less than one minute. A second dipping and drying step may be used to thicken the coating and to hydrolyze any unhydrolyzed ester functionalities, although any unhydrolyzed ester goups may react with functionalities in the preservative coating.

To form a preservative coating, a monomer such as a vinyl acetate derivative may be mixed with other monomers in a solvent such as isopropyl alcohol as seen at block 430. The solvent used with the drug-polymer preservative coating may be selected such that the barrier coating is not dissolved in the drug-polymer solvent. The mixture may be reacted to form a polymer, and one or more bioactive agents may be mixed with the polymerized mixture to form a drug polymer with a predefined elution rate as seen at block 440. A suitable bioactive agent or a solution containing the bioactive agent may be mixed in with the solution up to 75 percent bioactive agent or greater by weight in the drug-polymer coating. Alternatively, a polymer such as a copolyester or block copolymer may be dissolved in a suitable solvent, and one or more bioactive agents may be added to the mixture as seen at blocks 430 and 440.

One or more preservatives may be selected and added to the mixture as seen at block 450. The preservatives in the drug-polymer coating may comprise between 0.01 percent and 10 percent or higher of the preservative coating by weight. The preservative may be an antioxidant, such as BHT or vitamins A through E.

The drug polymer with the preservatives may be coated on a stent or stent framework, as seen at block 460. The drug polymer with the preservative may be applied to the stent by dipping, spraying, painting, or any other suitable method for applying the polymer, and then dried as seen at block 470. Drying of the primary coat to eliminate or remove any volatile components may be done at room temperature or elevated temperatures under dry nitrogen or other suitable environment. The thickness of the preservative coating may range between 1.0 microns and 200 microns, or greater in order to provide satisfactory pharmacological benefit with the bioactive agent.

A system for treating vascular conditions such as heart disease may be assembled using a catheter and a preservative-coated stent coupled to the catheter. The stent may be coated with a preservative coating with at least one preservative and optionally one or more interdispersed bioactive agents, and optionally coated with a barrier coating between the preservative coating and the stent framework. Finished coated stents may be reduced in diameter and placed into the distal end of the catheter, in a fashion to form an interference fit that secures the stent onto the catheter. The catheter with the stent may be placed in a catheter package and sterilized prior to shipping and storing. Sterilization using conventional means may be accomplished before clinical use.

Although the present invention applies to cardiovascular and endovascular stents with timed-release pharmaceutical drugs, the use of preservatives in polymer-drug coatings may be applied to other implantable and blood-contacting biomedical devices such as coated pacemaker leads, microdelivery pumps, feeding and delivery catheters, heart valves, artificial livers and other artificial organs.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. A system for treating a vascular condition, comprising:
a catheter;
a stent coupled to the catheter, the stent including a stent framework; and
a preservative coating operably disposed on the stent framework, wherein the preservative coating includes at least one antioxidant.

2. The system of claim 1 wherein the preservative coating comprises a drug polymer including a bioactive agent to provide a therapeutic characteristic.

3. The system of claim 2 wherein the bioactive agent is selected from the group consisting of an antineoplastic agent, an antiproliferative agent, an antisense agent, an antiplatelet agent, an antithrombogenic agent, an anticoagulant, an antibiotic, an anti-inflammatory agent, a gene therapy agent, an organic drug, a pharmaceutical compound, a recombinant DNA product, a recombinant RNA product, a collagen, a collagenic derivative, a protein, a protein analog, a saccharide, a saccharide derivative, and combinations thereof.

4. The system of claim 1 wherein the antioxidant comprises butylated hydroxytoluene.

5. The system of claim 1 wherein the antioxidant is selected from the group consisting of vitamin A, vitamin B, vitamin C, vitamin D, and vitamin E.

6. The system of claim 1 wherein the catheter includes a balloon used to expand the stent.

7. The system of claim 1 wherein the catheter includes a sheath that retracts to allow expansion of the stent.

8. The system of claim 1 wherein the stent framework comprises a metallic base.

9. The system of claim 8 wherein the metallic base is selected from the group consisting of stainless steel, nitinol, tantalum, MP35N alloy, a suitable biocompatible alloy, a suitable biocompatible material, and combinations thereof.

10. The system of claim 1 wherein the stent framework comprises a polymeric base.

11. The system of claim 1 further comprising:
a barrier coating interdisposed between the stent framework and the preservative coating.

12. The system of claim 11 wherein the barrier coating comprises parylene.

13. The system of claim 11 wherein the barrier coating comprises a silane coupling agent.

14. The system of claim 11 wherein the barrier coating has a thickness between 0.1 and 10 microns.

15. A preservative-coated stent, comprising:
a stent framework;
a polymeric coating on the stent framework; and
a preservative interdispersed within the polymeric coating

16. The preservative-coated stent of claim 15 wherein the preservative comprises at least one antioxidant selected from the group consisting of vitamin A, vitamin B, vitamin C, vitamin D, vitamin E, and butylated hydroxytoluene.

17. The preservative-coated stent of claim 15 further comprising:
a barrier coating interdisposed between the stent framework and the preservative coating.

18. The preservative-coated stent of claim 17 wherein the barrier coating is selected from the group consisting of parylene and a silane coupling agent.

19. A method of manufacturing a preservative-coated stent, comprising:
mixing a polymeric material with a solvent to form a polymeric mixture;
interdispersing a preservative in the polymeric mixture to form a preservative coating;
applying the preservative coating onto a stent framework; and
drying the preservative coating.

20. The method of claim 19 wherein the preservative comprises at least one antioxidant selected from the group consisting of vitamin A, vitamin B, vitamin C, vitamin D, vitamin E, and butylated hydroxytoluene.

21. The method of claim 19 further comprising:
applying a barrier coating onto the stent framework, wherein the barrier coating is applied prior to applying the preservative coating.

22. The method of claim 21 wherein the barrier coating is selected from the group consisting of parylene and a silane coupling agent.
